(19) United States
(12) Patent Application Publication
(10) Pub. No.: US 2015/0219673 A1
(43) Pub. Date: Aug. 6, 2015

(54) THE USE OF GENE EXPRESSION PROFILING AS A BIOMARKER FOR ASSESSING THE EFFICACY OF HDAC INHIBITOR TREATMENT IN NEURODEGENERATIVE CONDITIONS

(71) Applicant: Massachusetts Institute of Technology, Cambridge, MA (US)

(72) Inventors: Li-Huei Tsai, Cambridge, MA (US); Johannes Graeff, Waban, MA (US); Nadine F. Joseph, Chelsea, MA (US)

(21) Appl. No.: 14/425,519

(22) PCT Filed: Sep. 4, 2013
(86) PCT No.: PCT/US2013/058020

§ 371 Date: Mar. 3, 2015

Related U.S. Application Data
(60) Provisional application No. 61/696,426, filed on Sep. 4, 2012.

(51) Int. Cl. G01N 33/68; A61K 31/167; C12Q 1/68

(52) U.S. Cl. CPC G01N 33/6896; C12Q 1/6883; A61K 31/167; G01N 2800/2821; C12Q 2600/158; C12Q 2600/106; G01N 2800/52

(57) ABSTRACT

The present invention relates to methods, arrays, and kits for identifying Alzheimer's disease phenotype and for assessing the efficacy of putative AD therapies. In some aspects provided, is a method of identifying the presence of an Alzheimer's disease phenotype in a subject comprising: performing an assay to measure an expression pattern of at least one Alzheimer's associated gene.

### RELATED APPLICATION

This application claims the benefit under 35 U.S.C. §119(e) of U.S. Provisional Application No. 61/696,426, filed Sep. 4, 2012, the entire content of which is hereby incorporated by reference.

### FEDERALLY SPONSORED RESEARCH

This invention was made with government support under Grant No. NS078839 awarded by the National Institutes of Health. The government has certain rights in this invention.

### FIELD OF THE INVENTION

The present invention relates to methods, arrays and kits for diagnosing and monitoring Alzheimer's disease and assessing efficacy of treatment.

### BACKGROUND OF THE INVENTION

Alzheimer's disease (AD) is the leading cause of senile dementia worldwide, and leads to a marked loss in cognitive function, often reducing an afflicted person to an invalid state. AD has been estimated to afflict 5 to 11 percent of the population over age 65 and as much as 47 percent of the population over age 85. Moreover as adults born during the population boom of the 1940's and 1950's approach the age when AD becomes more prevalent, the control and treatment of AD will become an even more significant health care problem. However, to date there are no reliable methods to molecularly diagnose the disease or to monitor the efficacy of putative treatments.

### SUMMARY OF THE INVENTION

This invention relates in some aspects to methods, arrays and kits for diagnosing and monitoring Alzheimer's disease and assessing efficacy of treatment. In some aspects provided, is a method of identifying the presence of an Alzheimer's disease phenotype in a subject comprising: performing an assay to measure an expression pattern of at least one Alzheimer's disease-associated gene in an isolated biological sample from the subject; and comparing the expression pattern with an appropriate reference expression pattern of the at least one Alzheimer's disease-associated gene, wherein the results of the comparison are indicative of the presence of an Alzheimer's disease phenotype in the subject.

In another aspect provided, is a method of assessing the efficacy of a putative therapy for Alzheimer's disease in a subject in need thereof comprising obtaining a biological sample from the subject; administering the putative therapy to the subject to treat the Alzheimer's disease; measuring an expression pattern of at least one Alzheimer's disease-associated gene in the biological sample; and comparing the expression pattern with an appropriate reference expression pattern of the at least one Alzheimer's disease-associated gene, wherein the results of the comparison are indicative of the efficacy of the putative therapy. In certain embodiments of the invention, the expression pattern of at least 5, at least 10, at least 25, at least 50, at least 75, at least 100, at least 125, at least 150, at least 175, at least 200, at least 225, at least 250 Alzheimer's disease-associated genes is measured, and compared to the appropriate reference expression pattern. In certain embodiment of the invention, a biological sample is selected from the group consisting of blood, serum, cerebrospinal fluid, urine and tissue. In certain embodiments, the appropriate reference expression pattern comprises expression levels of the Alzheimer's disease-associated genes in a biological sample obtained from a subject who does not have Alzheimer's disease. In certain embodiment of the invention, the appropriate reference expression pattern comprises expression levels of the Alzheimer's disease-associated genes in a biological sample obtained from the subject prior to treatment. In certain embodiments, the appropriate reference expression pattern comprises standard expression levels of the Alzheimer's disease-associated genes. In certain embodiments, the expression pattern of Alzheimer's disease associated genes of the subject is monitored over time. In certain embodiments, the Alzheimer's associated genes are selected based on their differential expression pattern in a biological sample obtained from a subject who does not have Alzheimer's disease against a subject who has Alzheimer's disease. In certain embodiments, the Alzheimer's associated genes are selected from Table 1, 2, and/or 3. In some embodiments, the Alzheimer's associated genes comprise Tbc1d2, Tspan33, and/or Kit.

In certain embodiments, the expression pattern of RNA encoded by the Alzheimer's disease associated genes is measured using a hybridization-based assay. In a further embodiment, the hybridization-based assay is an oligonucleotide array assay, an oligonucleotide conjugated bead assay, a molecular inversion probe assay, a serial analysis of gene expression (SAGE) assay, or an RT-PCR assay.

In certain embodiments, the expression pattern of proteins encoded by the Alzheimer's disease associated genes is measured using an antibody-based assay. In certain embodiments, the antibody-based assay is an antibody array assay, an antibody conjugated-bead assay, an enzyme-linked immunosorbent (ELISA) assay or an immunoblot assay.

In certain embodiments, the putative therapy is an HDAC inhibitor.

In some aspects provided, the invention relates to an array comprising oligonucleotide probes that hybridize to nucleic acids having sequence correspondence to mRNA of at least 10 Alzheimer's disease-associated genes, wherein the Alzheimer's disease-associated genes are selected based on their differential expression pattern in a biological sample obtained from a subject who does not have Alzheimer's disease against a subject who has Alzheimer's disease.

In some aspects provided, the invention relates to an array comprising antibodies that bind specifically to proteins encoded by at least 10 Alzheimer's disease-associated genes, wherein the Alzheimer's disease-associated genes are selected based on their differential expression pattern in a biological sample obtained from a subject who does not have Alzheimer's disease against a subject who has Alzheimer's disease.

In some aspects provided, the invention is a method of monitoring progression of Alzheimer's disease in a subject in need thereof comprising obtaining a first biological sample from the subject; measuring a first expression pattern of at least one Alzheimer's disease-associated gene in the biological sample; obtaining a second biological sample from the subject; measuring a second expression pattern of the at least one Alzheimer's disease-associated gene in the biological sample; and comparing the first expression pattern with the second expression pattern, wherein the results of the comparison are indicative of the extent of progression of Alzheimer's disease in the subject. In certain embodiments, the subject is treated with HDAC inhibitor therapy between obtaining the first and the second biological sample. In certain embodiments, the time between obtaining the first biological sample and obtaining the second biological sample from the subject is a time sufficient for a change in severity of Alzheimer's disease to occur in the individual.

In some embodiments, the method is a method for identifying a therapy for the subject, and wherein the method involves selecting an HDAC inhibitor as a therapy for the subject if the Alzheimer's disease associated gene that is modulated is a gene from Table 2 or 3. In certain embodiments, the method further comprises treating the subject with an HDAC inhibitor. In certain embodiments, the HDAC inhibitor is CI-994.

In some aspects provided, the invention relates to a kit comprising a package housing including one or more containers with reagent for measuring an expression pattern of at least one Alzheimer's disease-associated gene from the biological sample and instructions for determining the expression patterns of the at least one Alzheimer's disease-associated gene and comparing the expression pattern with an appropriate reference expression pattern of the at least one Alzheimer's disease-associated gene. In certain embodiments, the reagent for measuring an expression pattern of at least one Alzheimer's disease-associated gene can be any of the arrays described herein.

According to some aspects of the invention, methods for treating a subject having Alzheimer's disease are provided. The methods comprise administering an inhibitor of an Alzheimer's disease gene upregulated in blood and brain to the subject in an amount effective to treat the subject. In some embodiments, the Alzheimer's disease gene upregulated in blood and brain is selected from the group consisting of Cdr2; Stk39; Tbc1d2; Bmp7; Nsdh1; Lbp; Tspan33; Cish; Fam46c; Cts1; Kit; Crtac1; Emilin1; Pafah2; Nqo1; Ptprf; and Ttc12.

In yet other aspects, the invention includes methods for treating inflammatory disorders of the brain and central nervous system (CNS). The method involves the administration of an HDAC inhibitor in an effective amount for treating the inflammatory disorder of the brain or CNS. In some embodiments the inflammatory disorder of the brain is an infectious agent associated disease such as encephalitis, Lyme's disease, abscess, meningitis, vasculitis, tropical spastic paraparesis, or cytomegalovirus (CMV) or human immunodeficiency virus (HIV) associated neuronal disease, or a non-cognitive neurodegenerative disease such as depression, multiple sclerosis, ADHD, ADD, anxiety, autism, Arachnoid cysts, Huntington's disease, Locked-in syndrome, Parkinson's disease, Tourette syndrome or bipolar disease.

In some embodiments the HDAC inhibitor is an HDAC2 inhibitor. The HDAC2 inhibitor may be a selective HDAC2 inhibitor. In other embodiments the HDAC2 inhibitor is non-selective but is not an HDAC1, HDAC5, HDAC6, HDAC7 and/or HDAC10 inhibitor. In yet other embodiments the HDAC2 inhibitor is an HDAC1/HDAC2 or an HDAC2/HDAC3 selective inhibitor or an HDAC1/HDAC2/HDAC3 selective inhibitor. In some embodiments the HDAC2 inhibitor is CI994.

In some embodiments the methods involve the measurement of inflammatory factors such as cytokines prior to, during and/or after treatment with the HDAC inhibitor. In some embodiments the inflammatory factors include at least one Alzheimer's disease-associated gene. In some embodiments the inflammatory factors are measured from a biological sample as described herein. The biological sample may be, for instance, blood or plasma.

In some aspects provided, the invention relates to a method of identifying the presence of an Alzheimer's disease phenotype in a subject. The method comprises performing an assay to measure a level of a beta-amyloid proteins in an isolated biological sample from the subject; and comparing the level of expression with an appropriate reference level of beta-amyloid proteins, wherein a lower level of beta-amyloid protein in the biological sample in comparison to a reference level associated with a normal subject is indicative of the presence of an Alzheimer's disease phenotype in the subject, and wherein the biological sample is a tissue other than the brain. In some embodiments, the biological sample is cerebrospinal fluid, blood or plasma.

Each of the embodiments and aspects of the invention can be practiced independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

FIG. 1 is a heatmap showing the expression levels of the 239 genes that are rescued by CI-994 treatment. These are the genes that are differentially expressed between SXFAD VEH, and SXFAD CI-994, but not differentially expressed between CON-VEH and SXFAD VEH (significance level p<0.05).

FIG. 2 is a heatmap showing 69 genes that are differentially expressed between wild type and SXFAD mice and rescued with CI-994 treatment. The 69 differentially expressed genes were identified by RNA-sequencing of PBMC of vehicle treated FAD, WT, and CI-994 treated FAD mice. Each line represents a differentially expressed gene and each row shows the gene expression averaged over two animals.

### DETAILED DESCRIPTION OF THE INVENTION

The present invention relates, in one aspect, to the discovery of biomarkers for diagnosing Alzheimer's disease (AD) and for testing the efficiency of putative treatments. In some embodiments, the present invention relates to methods for identifying the presence of AD phenotype in a subject. In some embodiments, methods to assess the efficacy of a putative therapy for AD in a subject are provided. In some embodiments, methods of monitoring the progression of AD in a subject are provided. In some embodiments, the present invention relates to arrays comprising oligonucleotides or antibodies that recognize mRNAs and proteins of AD-associated genes.

AD is a degenerative brain disorder characterized by cognitive and noncognitive neuropsychiatric symptoms, which accounts for approximately 60% of all cases of dementia for patients over 65 years old. In Alzheimer's disease the cognitive systems that control memory have been damaged. Often long-term memory is retained while short-term memory is lost; conversely, memories may become confused, resulting in mistakes in recognizing people or places that should be familiar. Psychiatric symptoms are common in Alzheimer's disease, with psychosis (hallucinations and delusions) present in many patients. The neuropathology is characterized by the formation of amyloid plaques and neurofibrillary tangles in the brain.

Over the past years, it has been discovered that epigenetic mechanisms in terms of posttranslational histone modifications, such as acetylation, and DNA methylation are deregulated during the progression of AD and substantially contribute to the AD-related cognitive decline. Acetylation neutralizes the positive charge of the lysine side chain of histones, and is thought to impact chromatin structure in a manner that facilitates transcription (e.g., by allowing transcription factors increased access to DNA). In vivo, the acetylation state of chromatin is thought to be maintained by a dynamic balance between the activities of enzymes, histone acetyl transferases (HATs) and histone deacetylases (HDACs). Different classes of small molecule inhibitors of HDACs have shown promising potential in rescuing cognitive capacities in AD-related animal models. For example, the HDAC inhibitor 4-(acetylamino)-N-(2-aminophenyl)benzamide (CI-994) and its metabolite dinaline have been shown to improve cognitive function in vivo, and can be used to treat AD (see US 2011/0224303).

It has been demonstrated experimentally using a mouse model of familial AD, the 5XFAD mice, that a number of genes are differentially expressed in 5XFAD mice as compared to control mice without AD. Moreover, it was also discovered according to the invention that HDAC inhibitor treatment of the 5XFAD mice rescued to near completion the differentially expressed genes in 5XFAD mice to levels comparable to control mice indicating that the HDAC inhibitor treatment reversed multiple aspects of AD at the molecular level.

As described herein, a variety of genes are differentially expressed in subjects having AD as compared to subjects identified as not having AD. An “Alzheimer's disease-associated gene” is a gene whose expression level is modulated in an Alzheimer disease subject compared to the expression level of the same gene in a subject not having Alzheimer's disease. The difference in expression levels is statistically significant. Examples of AD-associated genes include, but are not limited to, the genes listed in Table 1, 2 and/or 3. In some embodiments, the AD-associated genes include, but are not limited to, Arc, Atp2b3, Bsg, Cdr2, Cnst, Coro2b, Cpne7, Kit, Lingo 1, and Stk39. In some embodiments, the AD-associated genes include, but are not limited to, Adcy1, Cabp7, Cxcl14, Igfbp5, Npas4, and Ppp1r1a. In some embodiments, the AD-associated genes include, but are not limited to Tbc1d2, Tspan33, and/or Kit. In some embodiments, the AD-associated genes are not Lbp, Crtac1 and/or Nqo1.

Accordingly, some aspects of the invention relate to methods of identifying the presence of an Alzheimer's disease phenotype in a subject. The method comprises performing an assay to measure an expression pattern of at least one Alzheimer's disease-associated gene in an isolated biological sample from the subject, and comparing the expression pattern with an appropriate reference expression pattern of the at least one Alzheimer's disease-associated gene, wherein the results of the comparison are indicative of the presence of an Alzheimer's disease phenotype in the subject.

The methods disclosed herein may be used in combination with any one of a number of standard diagnostic approaches to identify AD in subjects, including but not limited to, mental status testing, physical and neurological exams, and brain imaging.

According to some aspects of the invention, methods of assessing the efficacy of a putative therapy for Alzheimer's disease in a subject are provided. The methods comprise obtaining a biological sample from the subject, administering the putative therapy to the subject to treat the Alzheimer's disease, measuring an expression pattern of at least one Alzheimer's disease-associated gene in the biological sample, and comparing the expression pattern with an appropriate reference expression pattern of the at least one Alzheimer's disease-associated gene, wherein the results of the comparison are indicative of the efficacy of the putative therapy.

In some embodiments, the putative therapy for AD includes, but is not limited to, administration of an HDAC inhibitor. In some embodiments, the HDAC inhibitor is 4-(acetylamino)-N-(2-aminophenyl)benzamide (CI-994), its metabolite dinaline or pharmaceutically acceptable salts, esters, or prodrugs thereof. The CI-994 or dinaline may be administered at a dosage effectively low to maintain a cumulative effective CI-994 or dinaline serum concentration. The CI-994 or dinaline may be administered orally, transdermally, intravenously, cutaneously, subcutaneously, nasally, intramuscularly, intraperitonealy, intracranially, or intracerebroventricularly.

According to some aspects of the invention, methods of monitoring progression of Alzheimer's disease in a subject are provided. The methods comprise obtaining a first biological sample from the subject, measuring a first expression pattern of at least one Alzheimer's disease-associated gene in the biological sample, obtaining a second biological sample from the subject, measuring a second expression pattern of the at least one Alzheimer's disease-associated gene in the biological sample, comparing the first expression pattern with the second expression pattern, wherein the results of the comparison are indicative of the extent of progression of Alzheimer's disease in the subject.

As used herein, a “subject” refers to any mammal, including humans and non-humans, such as primates. Typically the subject is a human. A subject in need of identifying the presence of AD phenotype is any subject at risk of, or suspected of, having AD. A subject at risk of having AD may be a subject having one or more risk factors for AD. Risk factors for AD include, but are not limited to, age, family history, heredity and brain injury. Other risk factors will be apparent the skilled artisan. A subject suspected of having AD may be a subject having one or more clinical symptoms of AD. A variety of clinical symptoms of AD are known in the art. Examples of such symptoms include, but are not limited to, memory loss, depression, anxiety, language disorders (eg, anomia) and impairment in their visuospatial skills.

In some embodiments, the subject has AD. In some embodiments, the subject has AD and is undergoing a putative treatment for AD. The methods described herein may be used to determine the efficacy of a putative therapy for AD, i.e., for evaluating the responsiveness of the subject to a putative therapy for AD. Based on this evaluation, the physician may continue the therapy, if there is a favorable response, or discontinue and change to another therapy if the response is unfavorable.

The methods disclosed herein typically involve determining expression pattern of at least one AD-associated gene in a biological sample isolated from a subject. The methods may involve determining expression levels of at least 5, at least 10, at least 25, at least 50, at least 75, at least 100, at least 125, at least 150, at least 175, at least 200, at least 225, at least 250 Alzheimer's disease-associated genes in a biological sample isolated from a subject.

The expression pattern of the AD-associated genes may be measured by performing an assay to determine the expression level of an RNA encoded by an Alzheimer's disease associated gene. Examples of assay to measure RNA levels include, but are not limited to hybridization-based assays. Hybridization-based assay are well known in the art, and include, but are not limited to, an oligonucleotide array assay (e.g., microarray assays), an oligonucleotide conjugated bead assay (e.g., Multiplex Bead-based Luminex® Assays), a molecular inversion probe assay, a serial analysis of gene expression (SAGE) assay, northern blot assay, an in situ hybridization assay, cDNA array assays, RNase protein assays, or an RT-PCR assay. Multiplex systems, such as oligonucleotide arrays or bead-based nucleic acid assay systems are particularly useful for evaluating levels of a plurality of nucleic acids in simultaneously. RNA-Seq (mRNA sequencing using Ultra High throughput or Next Generation Sequencing) may also be used to determine expression levels. Other appropriate methods for determining levels of nucleic acids will be apparent to the skilled artisan.

The expression pattern of the AD-associated genes may be determined as the level of protein encoded by the genes. Examples of assays to measure protein levels include, but are not limited to, antibody-based assays. Antibody-based assays are well known in the art and include, but are not limited to, antibody array assays, antibody conjugated-bead assays, enzyme-linked immuno-sorbent (ELISA) assays, immunofluorescence microscopy assays, and immunoblot assays. Other methods for determining protein levels include mass spectroscopy, spectrophotometry, and enzymatic assays. Still other appropriate methods for determining levels of proteins will be apparent to the skilled artisan.

The methods may involve obtaining a biological sample from the subject. As used herein, the phrase “obtaining a biological sample” refers to any process for directly or indirectly acquiring a biological sample from a subject. For example, a clinical sample may be obtained (e.g., at a point-of-care facility, e.g., a physician's office, a hospital) by procuring a tissue or fluid sample (e.g., blood draw, spinal tap) from an individual. Alternatively, a biological sample may be obtained by receiving the biological sample (e.g., at a laboratory facility) from one or more persons who procured the sample directly from the individual.

In some embodiments, a first and second biological sample is obtained from the subject. In some embodiments, the subject is treated with a putative therapy for AD in the time between obtaining the first biological sample and obtaining the second biological sample from the subject. In some embodiments, the time between obtaining the first biological sample and obtaining the second biological sample the subject is a time sufficient for a change in severity of Alzheimer's disease to occur in the individual.

The term “biological sample” refers to a sample derived from a subject, e.g., a patient. Biological samples include, but are not limited to tissue (e.g., brain tissue), cerebrospinal fluid, blood, blood fractions (e.g., serum, plasma), sputum, fine needle biopsy samples, urine, peritoneal fluid, and pleural fluid, or cells therefrom (e.g., blood cells (e.g., white blood cells, red blood cells)). Accordingly, a biological sample may comprise a tissue, cell or biomolecule (e.g., RNA, protein). In some embodiments, the biological sample is a sample of peripheral blood, serum, cerebrospinal fluid, urine and tissue.

It is to be understood that a biological sample may be processed in any appropriate manner to facilitate determining expression levels of AD-associated genes. For example, biochemical, mechanical and/or thermal processing methods may be appropriately used to isolate a biomolecule of interest, e.g., RNA, protein, from a biological sample. A RNA sample may be isolated from a clinical sample by processing the biological sample using methods well known in the art and levels of an RNA encoded by an AD-associated gene may be determined in the RNA sample. A protein sample may be isolated from a clinical sample by processing the clinical sample using methods well known in the art, and levels of a protein encoded by an AD-associated gene may be determined in the protein sample. The expression levels of AD-associated genes may also be determined in a biological sample directly.

The methods disclosed herein also typically comprise comparing expression pattern of AD-associated genes with an appropriate reference expression pattern. An appropriate reference expression pattern can be determined or can be a pre-existing reference expression pattern. An appropriate reference expression pattern may be a threshold expression level of an AD-associated gene such that an expression level that is above or below the threshold level is indicative of AD in a subject. In some embodiments, the appropriate reference expression pattern comprises standard expression levels of the Alzheimer's disease-associated genes.

An appropriate reference expression pattern may be an expression pattern indicative of a subject that is free of AD. For example, an appropriate reference expression pattern may be representative of the expression level of a particular AD-associated gene in a biological sample obtained from a subject who does not have AD. When an appropriate reference expression pattern is indicative of a subject who does not have AD, a significant difference between an expression pattern determined from a subject in need of diagnosis or monitoring of AD and the appropriate reference expression pattern may be indicative of AD in the subject. Alternatively, when an appropriate reference expression pattern is indicative of the subject being free of AD, a lack of a significant difference between an expression pattern determined from a subject in need of diagnosis or monitoring of AD and the appropriate reference expression pattern may be indicative of the individual being free of AD.

An appropriate reference level may be an expression pattern indicative of AD. For example, an appropriate reference expression pattern may be representative of the expression pattern of an AD-associated gene in a biological sample obtained from a subject known to have AD. When an appropriate reference expression pattern is indicative of AD, a lack of a significant difference between an expression pattern determined from a subject in need of diagnosis and monitoring of AD and the appropriate reference expression pattern may be indicative of AD in the subject. Alternatively, when an appropriate reference expression pattern is indicative of AD, a significant difference between an expression pattern determined from a subject in need of diagnosis or monitoring of AD and the appropriate reference expression pattern may be indicative of the subject being free of AD.

An appropriate reference expression pattern may also comprise expression levels of the Alzheimer's disease-associated genes in a biological sample obtained from the subject prior to administration of a putative therapy for AD. In some embodiments, the expression pattern of AD-associated genes of the subject is monitored over time.

The magnitude of difference between an expression pattern and an appropriate reference expression pattern may vary. For example, a significant difference that indicates diagnosis or progression of AD may be detected when the expression level of an AD-associated gene in a biological sample is at least 1%, at least 5%, at least 10%, at least 25%, at least 50%, at least 100%, at least 250%, at least 500%, or at least 1000% higher, or lower, than an appropriate reference level of that gene. Similarly, a significant difference may be detected when the expression level of an AD-associated gene in a biological sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the appropriate reference level of that gene. Significant differences may be identified by using an appropriate statistical test. Tests for statistical significance are well known in the art and are exemplified in Applied Statistics for Engineers and Scientists by Petruccelli, Chen and Nandram 1999 Reprint Ed.

It is to be understood that a plurality of expression levels may be compared with plurality of appropriate reference levels, e.g., on a gene-by-gene basis, as a vector difference, in order to assess the AD status of the subject or the efficacy of a putative treatment being administered to the subject. In such cases, Multivariate Tests, e.g., Hotelling's T2 test, may be used to evaluate the significance of observed differences. Such multivariate tests are well known in the art and are exemplified in Applied Multivariate Statistical Analysis by Richard Arnold Johnson and Dean W. Wichern Prentice Hall; 4th edition (Jul. 13, 1998).

According to some aspects of the invention, methods for identifying a therapy for a subject are provided. The methods comprise selecting an HDAC inhibitor as a therapy for the subject if the Alzheimer's disease associated gene that is modulated is a gene from Table 2 or 3. In some embodiments, the methods further comprise treating the subject with an HDAC inhibitor. In some embodiments, the HDAC inhibitor is CI-994.

According to some aspects of the invention, methods for treating a subject having Alzheimer's disease are provided. The methods comprise administering an inhibitor of an Alzheimer's disease gene upregulated in blood and brain to the subject in an amount effective to treat the subject. In some embodiments, the Alzheimer's disease gene upregulated in blood and brain is selected from the group consisting of Cdr2; Stk39; Tbc1d2; Bmp7; Nsdh1; Lbp; Tspan33; Cish; Fam46c; Cts1; Kit; Crtac1; Emilin1; Pafah2; Nqo1; Ptprf; and Ttc12.

Thus, in some aspects the specific Alzheimer's disease genes or corresponding proteins identified herein may be utilized as a therapeutic target. These genes/proteins can be targeted by specific reagents designed to interfere with their functions and or expression. For example many of the proteins corresponding to the Alzheimer's disease genes have specific receptors and therapeutic agents can be used to block the interactions of these proteins with their receptors or with other proteins in order to treat Alzheimer's disease. Additionally, some of the proteins corresponding to the Alzheimer's disease genes are enzymes. Therapeutics may be used to interfere with the enzymatic activities of these proteins. Additionally, the expression of these Alzheimer's disease genes can be inhibited using inhibitory RNA, particularly when the RNA can be targeted to the brain tissue as well as the peripheral blood. A therapeutic agent useful for blocking a protein-receptor or a protein-protein interaction is any type of reagent that binds to one or both of the proteins (receptor or ligand) and blocks the proteins from interacting. The reagent may be a protein, small molecule, nucleic acid or any other type of molecule which binds to and blocks the interaction, such as a receptor antagonist. For example the reagent may be (using antibodies, antibody fragments, peptides or peptidomimetics.

A therapeutic agent useful for blocking enzyme function is any reagent that interrupts the interaction or activity of the enzyme with it's substrate. For example the reagent may directly interfere with the interaction. For instance a structural antagonist of the substrate may compete for binding to the enzyme and block the interaction between the enzyme and substrate. Additionally the regent may indirectly interfere with the interaction by causing a conformational change or stability change in the enzyme which results in a loss of the enzymes ability to bind to the substrate or act on the substrate.

Methods for inhibiting the expression of Alzheimer's disease genes described herein are known in the art. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA or shRNA-mir expression constructs) are used to reduce expression of a gene encoding any of the Alzheimer's disease genes described herein.

The inhibitors are administered in an effective amount. An effective amount is a dose sufficient to provide a medically desirable result and can be determined by one of skill in the art using routine methods. In some embodiments, an effective amount is an amount which results in any improvement in the condition being treated. In some embodiments, an effective amount may depend on the type and extent of Alzheimer's disease being treated and/or use of one or more additional therapeutic agents. However, one of skill in the art can determine appropriate doses and ranges of inhibitors to use, for example based on in vitro and/or in vivo testing and/or other knowledge of compound dosages.

When administered to a subject, effective amounts of the inhibitor will depend, of course, on the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

In the treatment of Alzheimer's disease, an effective amount is that amount which slows the progression of the disease, halts the progression of the disease, or reverses the progression of the disease. An effective amount includes that amount necessary to slow, reduce, inhibit, ameliorate or reverse one or more symptoms associated with Alzheimer's disease. In some embodiments, such terms refer to an improvement in memory function, and reading and writing skills.

An effective amount of a compound typically will vary from about 0.001 mg/kg to about 1000 mg/kg in one or more dose administrations, for one or several days (depending of course of the mode of administration and the factors discussed above). Actual dosage levels of the inhibitor can be varied to obtain an amount that is effective to achieve the desired therapeutic response for a particular patient, compositions, and mode of administration. The selected dosage level depends upon the activity of the particular compound, the route of administration, the tissue being treated, and prior medical history of the patient being treated. However, it is within the skill of the art to start doses of the inhibitor at levels lower than required to achieve the desired therapeutic effort and to gradually increase the dosage until the desired effect is achieved.

Described herein are oligonucleotide (nucleic acid) arrays that are useful in the methods for determining levels of multiple nucleic acids simultaneously. Also, described herein are antibody arrays that are useful in the methods for determining levels of multiple proteins simultaneously. Such arrays may be obtained or produced from commercial sources. Methods for producing nucleic acid arrays are well known in the art. For example, nucleic acid arrays may be constructed by immobilizing to a solid support large numbers of oligonucleotides, polynucleotides, or cDNAs capable of hybridizing to nucleic acids corresponding to mRNAs, or portions thereof. The skilled artisan is also referred to Chapter 22 “Nucleic Acid Arrays” of Current Protocols In Molecular Biology (Eds. Ausubel et al. John Wiley and #38; Sons NY, 2000), International Publication WO00/58516, U.S. Pat. No. 5,677,195 and U.S. Pat. No. 5,445,934 which provide non-limiting examples of methods relating to nucleic acid array construction and use in detection of nucleic acids of interest. In some embodiments, the nucleic acid arrays comprise, or consist essentially of, binding probes for mRNAs of at least 2, at least 5, at least 10, at least 20, at least 50, at least 100, at least 200, at least 300, or more genes selected from Table 1.

Methods for producing antibody arrays are also well known in the art. For example, antibody arrays may be constructed by fixing a collection of antibodies on a solid surface such as glass, plastic or silicon chip, for the purpose of detecting antigens. The skilled artisan is also referred to Rivas L A, García-Villadangos M, Moreno-Paz M, Cruz-Gil P, Gómez-Elvira J, Parro V (November 2008) “A 200-antibody microarray biochip for environmental monitoring: searching for universal microbial biomarkers through immunoprofiling”. Anal. Chem. 80 (21): 7970-9 and Chaga G S (2008). “Antibody arrays for determination of relative protein abundances”. Methods Mol. Biol. 441: 129-51, which provide non-limiting examples of methods relating to antibody array construction and use in detection of proteins of interest. In some embodiments, the antibody arrays comprise, or consist essentially of, antibodies for proteins of at least 2, at least 5, at least 10, at least 20, at least 50, at least 100, at least 200, at least 300, or more genes selected from Table 1.

Kits comprising reagents for measuring an expression pattern of at least one Alzheimer's disease-associated gene from the biological sample are also provided. Kits may include a package housing one or more containers with reagent for measuring an expression pattern of at least one Alzheimer's disease-associated gene from the biological sample and instructions for determining the expression patterns of the at least one Alzheimer's disease-associated gene and comparing the expression pattern with an appropriate reference expression pattern of the at least one Alzheimer's disease-associated gene. Kits comprising the oligonucleotide and antibody arrays described herein are also included.

Methods for treating inflammatory disorders of the brain and central nervous system (CNS) by administering an HDAC inhibitor are also part of the invention. An inflammatory disorder of the brain or CNS is a disease associated with inflammation in the brain or CNS tissues. In some instances it is a disease caused by or associated with an infectious agent. Examples of diseases caused by or associated with an infectious agent include but are not limited to encephalitis, abscess, meningitis, vasculitis, tropical spastic paraparesis, and cytomegalovirus (CMV) and human immunodeficiency virus (HIV) associated neuronal disease. In other instances the inflammatory disorder of the brain or CNS is a non-cognitive neurodegenerative disease associated with inflammation in the brain or CNS tissues. Examples of these types of diseases include but are not limited to depression, multiple sclerosis, ADHD, ADD, anxiety, autism, Arachnoid cysts, Huntington's disease, Locked-in syndrome, Parkinson's disease, Tourette syndrome, schizophrenia and bipolar disease. In some embodiments the inflammatory disorder of the brain or CNS is not a cognitive neurodegenerative disease such as Alzheimer's disease.

Brain abscesses may result from bacterial, fungal or viral infection. Examples of fungal infections include coccidioidomycosis, aspergillosis, Cysticercosis, and Neurocysticercosis. Bacterial infections include bacterial meningitis arising from Hemophilus influenza, Neisseria meningitides (Meningococcus) and Streptococcus pneumonia and sarcoidosis. Encephalitis results from arthropod-borne arboviruses (Eastern and Western equine encephalitis, St. Louis encephalitis, California virus encephalitis) and West Nile virus. The enteroviruses, such as coxsackie-virus and echoviruses, can produce a meningoencephalitis, but a more benign aseptic meningitis is more common with these organisms. Herpes simplex virus causes a severe form of acute encephalitis. Lyme Disease associated with Borrelia burgdorferi is also an inflammatory disease of the brain or CNS. Other infectious agents include Toxoplasma, Listeria, Treponema, Rubella, Cytomegalovirus, and Herpes simplex type 2. Cryptococcosis and Pogressive Multifocal Leukoencephalopathy (PML) are associated with HIV.

The inflammatory disorder of the brain or CNS which are non-cognitive neurodegenerative disorders have unique and distinct symptoms, but each is associated with inflammation. The methods of the invention reduce brain and CNS inflammation and are therefore useful for treating this group of disorders. Arachnoid cysts are often results in headache, seizures, ataxia (lack of muscle control), hemiparesis, macrocephaly and ADHD. Huntington's disease is a degenerative neurological disorder resulting in a progressive decline associated with abnormal movements. Locked-in syndrome associated with excessive inflammation causes physical but not cognitive paralysis. Parkinson's disease is associated with bradykinesia (slow physical movement), muscle rigidity, and tremors. Tourette's syndrome is a neurological disorder, associated with physical tics and verbal tics. Multiple sclerosis is a chronic, inflammatory demyelinating disease, involving visual and sensation problems, muscle weakness, and depression.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLE

### Example 1

To test if high-throughput genome-wide RNA sequencing can be readily used a biomarker for HDAC inhibitor-mediated treatment of cognitive decline associate with AD, a mouse model of familial AD, the SXFAD mice were used. These mice harbor point mutations in the AD-related pathogenic presenilin and amyloid precursor protein pathways, and recapitulate the majority of human AD pathologies, including amyloid-β deposition, neurodegeneration, and cognitive impairments.

Adult male SXFAD mice were treated chronically, i.e., for one month, with daily intraperitoneal injections of the histone deacetylase inhibitor CI-994 (1 mg/kg), which had been shown to reduce AD-related cognitive impairments. After completion of treatment, mice were sacrificed, their brain regions dissected, and total RNA extracted of the hippocampus, a brain region important for memory formation and storage. The RNA was quality controlled using Agilent's bioanalyzer 5′ and 3′-end labeled and sequenced on an Illumina HiSeq sequencer with 200 million reads per sample. Sequence reads were aligned to the mouse genome, and quality-filtered. Differential analysis was then conducted using Cuffdiff with IIlumina iGenome mm9 UCSC gene annotation. A total of 3 SXFAD samples were treated with CI-994 (SXFAD CI-994), 3 SXFAD samples were treated with saline (SXFAD VEH) and 3 control littermates (CON VEH) treated with saline were processed.

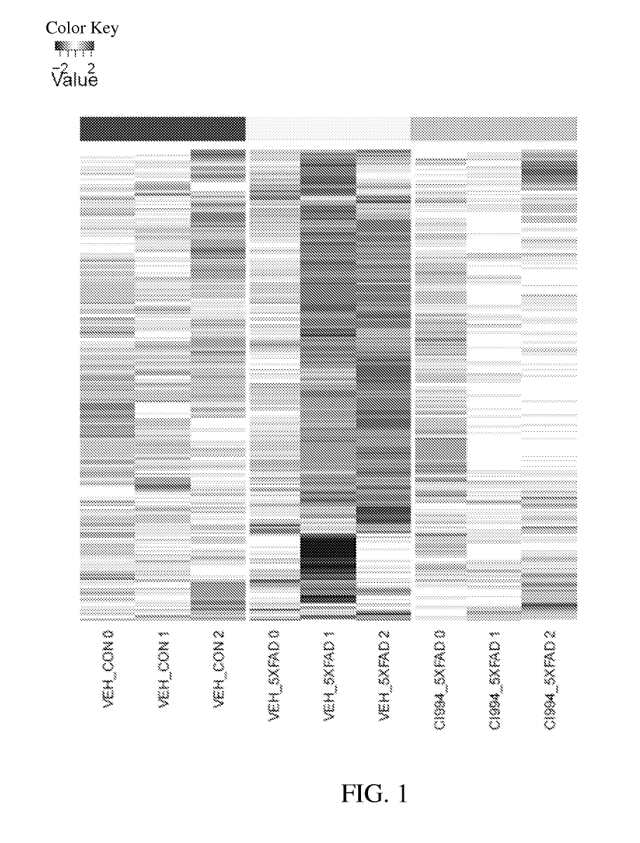

In the SXFAD mice treated with saline, the majority of differentially expressed genes were upregulated, although there were a subset of genes that were downregulated. As shown in FIG. 1, RNA sequencing revealed that CI-994 of the SXFAD mice rescued to near completion the differentially expressed genes in SXFAD mice to levels comparable to control mice indicating that CI-994 reversed multiple aspects of AD at the molecular level. In particular, the rescue of the downregulated genes by CI994 was the most complete (100%). Importantly, these results also demonstrate that CI-994 is not only symptom modifying, but is also disease modifying.

### Example 2

To test the potential of HDAC inhibitors as a novel disease-modifying approach against AD-related pathologies, two mouse models of AD-related pathologies, the CK-p25 and SXFAD were used. The former exhibits severe cognitive defects, alongside with profound neuronal loss and the presence of astrogliosis, beta-amyloid plaques and neurofibrillary tangles. The latter shows substantial cognitive decline, astogliosis and beta-amyloid deposition.

Chronic treatment with different HDAC inhibitors not only ameliorated cognitive deficits in both mouse models, but also reduced the amyloid burden in their brains, thereby demonstrating HDAC inhibitor treatment as a valuable disease modifying strategy.

TABLE 1

| Rescue | | CON- | 5XFAD- | 5XFAD- |
|---|---|---|---|---|
| Gene | Product | VEH | VEH | CI994 |
| 1700026L06Rik | uncharacterized protein C9orf9 | 0.624239 | 2.51472 | 0.562431 |
| | homolog | | | |
| 4833427G06Rik | UPF0722 protein C11orf88 | 0.586386 | 3.15463 | 1.15558 |
| | homolog | | | |
| Abhd2 | abhydrolase domain-containing | 15.6932 | 27.4262 | 16.6349 |
| | protein 2 | | | |
| Acaa2 | 3-ketoacyl-CoA thiolase, | 13.3945 | 26.0835 | 15.6611 |
| | mitochondrial | | | |
| Acacb | acetyl-Coenzyme A carboxylase | 0.617337 | 1.39029 | 0.666905 |
| | beta precursor | | | |
| Acss3 | acyl-CoA synthetase short-chain | 0.724694 | 1.88239 | 0.864404 |
| | family member 3, mitochondrial | | | |
| Adcy1 | adenylate cyclase type 1 | 138.844 | 79.0004 | 121.693 |
| Aebp1 | adipocyte enhancer-binding | 2.92188 | 6.26373 | 4.33426 |
| | protein 1 precursor | | | |
| Aldh1a1 | retinal dehydrogenase 1 | 17.0089 | 9.36183 | 16.8375 |
| Aldh2 | aldehyde dehydrogenase, | 33.598 | 48.5925 | 35.3667 |
| | mitochondrial precursor | | | |
| Als2cr4 | N/A | 12.0086 | 24.844 | 14.5748 |
| Angptl2 | angiopoietin-related protein 2 | 0.891048 | 4.2822 | 1.51893 |
| | precursor | | | |
| Antxr1 | anthrax toxin receptor 1 | 3.22803 | 6.56718 | 4.10607 |
| Apln | apelin precursor | 9.76795 | 6.12249 | 9.12137 |
| Arc | activity-regulated cytoskeleton- | 66.4138 | 48.1849 | 77.622 |
| | associated protein | | | |
| Arhgap28 | rho GTPase-activating protein 28 | 0.127921 | 0.510226 | 0.180021 |
| Arsg | arylsulfatase G precursor | 11.8198 | 20.3 | 13.3427 |
| Atf4 | cyclic AMP-dependent | 63.2085 | 81.1329 | 63.7327 |
| | transcription factor ATF-4 | | | |
| Atp10d | probable phospholipid- | 0.529973 | 2.31843 | 0.853749 |
| | transporting ATPase VD | | | |
| | precursor | | | |
| Atp11c | probable phospholipid- | 2.33689 | 6.57379 | 3.64937 |
| | transporting ATPase 11C isoform | | | |
| | b | | | |
| Atp2b3 | plasma membrane calcium- | 38.3395 | 60.8575 | 41.3436 |
| | transporting ATPase 3 | | | |
| Atp7a | ATPase, Cu++ transporting, | 1.49419 | 3.26003 | 1.98727 |
| | alpha polypeptide | | | |
| B230217C12Rik | uncharacterized protein | 43.4852 | 31.7014 | 41.8821 |
| | LOC68127 | | | |
| BC049635 | transmembrane protein | 0.0540868 | 1.37492 | 0.51401 |
| | ENSP00000340100 homolog | | | |
| Baiap2l1 | brain-specific angiogenesis | 0.273616 | 2.33057 | 0.622386 |
| | inhibitor 1-associated protein 2- | | | |
| | like protein 1 | | | |
| Bcam | basal cell adhesion molecule | 4.24441 | 8.05677 | 5.37994 |
| | precursor | | | |
| Bmp6 | bone morphogenetic protein 6 | 4.02641 | 11.1645 | 5.71336 |
| | precursor | | | |
| Bmp7 | bone morphogenetic protein 7 | 2.45079 | 6.25389 | 2.93075 |
| | precursor | | | |
| Brwd3 | bromodomain and WD repeat- | 1.68117 | 2.7935 | 1.78606 |
| | containing protein 3 | | | |
| Bsg | basigin, isoform C | 259.511 | 403.804 | 282.696 |
| Bst2 | bone marrow stromal antigen 2 | 3.2881 | 11.7966 | 4.30741 |
| | precursor | | | |
| Btbd3 | BTB/POZ domain-containing | 36.8885 | 23.5521 | 34.9585 |
| | protein 3 | | | |
| C1ql2 | complement C1q-like protein 2 | 43.6231 | 26.5977 | 39.6924 |
| | precursor | | | |
| C1qtnf5 | complement C1q tumor necrosis | 11.9862 | 35.9883 | 15.0312 |
| | factor-related protein 5 precursor | | | |
| C230081A13Rik | pseudopodium-enriched atypical | 9.29364 | 12.5865 | 8.24125 |
| | kinase 1 | | | |
| C530008M17Rik | uncharacterized protein | 17.2628 | 22.2054 | 17.1167 |
| | KIAA1211 | | | |
| Cabp7 | calcium-binding protein 7 | 208.995 | 159.797 | 205.981 |
| Car14 | carbonic anhydrase 14 precursor | 4.78747 | 14.7654 | 6.58424 |
| Ccdc141 | coiled-coil domain containing | 2.03824 | 3.97679 | 2.31681 |
| | 141 | | | |
| Ccnd1 | G1/S-specific cyclin-Dl | 17.2602 | 11.6935 | 15.4148 |
| Cdh3 | cadherin 3 precursor | 0.0235528 | 1.08593 | 0.368562 |
| Cdr2 | cerebellar degeneration-related | 4.31589 | 11.4284 | 5.99428 |
| | protein 2 | | | |
| Cndp1 | beta-Ala-His dipeptidase | 0.0995992 | 0.828409 | 0.251373 |
| Cnst | consortin | 9.93687 | 14.2815 | 10.5531 |
| Col17a1 | collagen alpha-1(XVII) chain | 0.012258 | 0.249454 | 0.0235052 |
| Col18a1 | collagen, type XVIII, alpha 1 | 0.641598 | 2.3876 | 0.783611 |
| | precursor | | | |
| Col4a3 | collagen alpha-3(IV) chain | 0.0646655 | 0.396931 | 0.145896 |
| | precursor | | | |
| Col4a4 | collagen, type IV, alpha 4 | 0.132942 | 0.479793 | 0.17018 |
| Coro2b | coronin-2B | 59.395 | 76.6161 | 59.6533 |
| Cpn1 | carboxypeptidase N catalytic | 0.0811035 | 0.793664 | 0.238107 |
| | chain precursor | | | |
| Cpne7 | copine-7 | 72.6339 | 103.506 | 72.8282 |
| Cpt1b | carnitine O-palmitoyltransferase | 0 | 0.551678 | 0 |
| | 1, muscle isoform | | | |
| Crb3 | crumbs protein homolog 3 | 0.291716 | 2.77759 | 0.654551 |
| | precursor | | | |
| Crhr2 | corticotropin-releasing factor | 0.595531 | 2.23327 | 0.6462 |
| | receptor 2 precursor | | | |
| Crtac1 | cartilage acidic protein 1 | 32.069 | 43.6956 | 33.2962 |
| | precursor | | | |
| Crtap | cartilage-associated protein | 2.37047 | 4.38652 | 2.38191 |
| | precursor | | | |
| Ctnnal1 | alpha-catulin | 3.47457 | 7.98776 | 4.53785 |
| Cul4b | cullin-4B | 12.5653 | 21.7765 | 14.4206 |
| Cxcl14 | C-X-C motif chemokine 14 | 69.3377 | 48.3092 | 62.1186 |
| | precursor | | | |
| Dab2 | disabled homolog 2 | 1.99778 | 4.72126 | 2.76509 |
| Dclk3 | serine/threonine-protein kinase | 6.04751 | 4.30744 | 6.88298 |
| | DCLK3 | | | |
| Dcn | decorin precursor | 17.6465 | 26.1779 | 18.2706 |
| Ddr2 | discoidin domain-containing | 1.51514 | 2.53485 | 1.21668 |
| | receptor 2 precursor | | | |
| Dgkh | | 8.88537 | 6.08947 | 8.27716 |
| Dio2 | type II iodothyronine deiodinase | 16.7737 | 9.34195 | 13.4167 |
| Dmrt3 | doublesex- and mab-3-related | 0.645078 | 1.9143 | 0.669161 |
| | transcription factor 3 | | | |
| Dnahc11 | dynein, axonemal, heavy chain 11 | 0.179947 | 0.418196 | 0.218072 |
| Doc2b | double C2-like domain- | 34.6523 | 24.2573 | 33.8561 |
| | containing protein beta | | | |
| Dpep1 | dipeptidase 1 precursor | 0.0846536 | 0.520015 | 0.139907 |
| Dpp7 | dipeptidyl peptidase 2 precursor | 7.36265 | 12.6549 | 8.54167 |
| Dsg2 | desmoglein-2 precursor | 0.848692 | 1.66813 | 0.999317 |
| Dsp | desmoplakin | 8.29833 | 4.89359 | 8.25841 |
| Ephx1 | epoxide hydrolase 1 precursor | 14.6948 | 25.3052 | 17.8668 |
| Eps8l2 | epidermal growth factor receptor | 1.25456 | 2.6724 | 1.37889 |
| | kinase substrate 8-like protein 2 | | | |
| F11r | junctional adhesion molecule A | 3.24994 | 6.67645 | 3.43792 |
| | precursor | | | |
| Fads2 | fatty acid desaturase 3 | 7.85965 | 12.5136 | 8.70066 |
| Fam163b | uncharacterized protein | 65.9083 | 43.1459 | 67.8893 |
| | LOC685169 | | | |
| Fam38a | piezo-type mechanosensitive ion | 0.61923 | 1.5668 | 0.661317 |
| | channel component 1 | | | |
| Fras1 | extracellular matrix protein | 0.639782 | 1.10392 | 0.748983 |
| | FRAS1 precursor | | | |
| Fst | frost | 1.83579 | 0.698451 | 1.75528 |
| Fxyd1 | phospholemman precursor | 32.7404 | 67.0955 | 37.5709 |
| Fzd4 | frizzled-4 precursor | 3.07798 | 6.22743 | 3.61315 |
| Fzd7 | frizzled-7 precursor | 3.6506 | 6.36982 | 4.15103 |
| Gabra2 | gamma-aminobutyric acid | 45.8025 | 70.6867 | 45.2624 |
| | receptor subunit alpha-2 | | | |
| | precursor | | | |
| Galm | aldose 1-epimerase | 1.35915 | 3.0491 | 1.68712 |
| Gas6 | growth arrest-specific protein 6 | 32.0329 | 56.7613 | 39.2248 |
| | precursor | | | |
| Glb1l2 | beta-galactosidase-1-like protein 2 | 0.372625 | 1.3626 | 0.548308 |
| Glul | glutamine synthetase | 274.777 | 177.415 | 251.125 |
| Gm11744 | progressive rod-cone | 1.29786 | 5.55272 | 2.59425 |
| | degeneration protein homolog | | | |
| | precursor | | | |
| Gm221 | coiled-coil domain-containing | 0.369657 | 1.23218 | 0.498957 |
| | protein C6orf97 | | | |
| Gm853 | ornithine decarboxylase-like | 0 | 0.191486 | 0 |
| Gng7 | guanine nucleotide-binding | 76.6674 | 49.2731 | 64.066 |
| | protein G(I)/G(S)/G(O) subunit | | | |
| | gamma-7 | | | |
| Gprc5c | G-protein coupled receptor | 0.932285 | 2.65099 | 1.25422 |
| | family C group 5 member C | | | |
| | isoform a precursor | | | |
| Grm2 | metabotropic glutamate receptor | 13.9183 | 9.77447 | 13.2878 |
| | 2 precursor | | | |
| Gyltl1b | glycosyltransferase-like protein | 0.118366 | 1.2708 | 0.192026 |
| | LARGE2 | | | |
| Hapln1 | hyaluronan and proteoglycan link | 12.1284 | 7.9551 | 10.6547 |
| | protein 1 precursor | | | |
| Hbb-b2 | hemoglobin subunit beta-2 | 5.7847 | 19.154 | 3.71396 |
| Hemk1 | hemK methyltransferase family | 3.9302 | 8.20469 | 4.07362 |
| | member 1 | | | |
| Homer2 | homer protein homolog 2 | 16.5669 | 10.5395 | 14.9404 |
| Hsd11b1 | cortico steroid 11-beta- | 31.0186 | 20.2365 | 28.1849 |
| | dehydrogenase isozyme 1 | | | |
| Hspg2 | basement membrane-specific | 0.636205 | 1.09737 | 0.737224 |
| | heparan sulfate proteoglycan core | | | |
| | protein precursor | | | |
| Ifi27l1 | interferon, alpha-inducible | 33.1532 | 55.4739 | 36.0763 |
| | protein 27 like 1 isoform 2 | | | |
| Igfbp5 | insulin-like growth factor-binding | 38.0494 | 29.4934 | 38.2003 |
| | protein 5 precursor | | | |
| Igfbp7 | insulin-like growth factor-binding | 16.7404 | 30.5241 | 18.1462 |
| | protein 7 precursor | | | |
| Igfn1 | immunoglobulin-like and | 0.0400676 | 0.407576 | 0.127126 |
| | fibronectin type III domain- | | | |
| | containing protein 1 | | | |
| Iqgap1 | ras GTPase-activating-like | 2.89759 | 4.31298 | 3.03842 |
| | protein IQGAP1 | | | |
| Isoc1 | isochorismatase domain- | 13.6633 | 10.1434 | 14.1426 |
| | containing protein 1 | | | |
| Kcnj10 | ATP-sensitive inward rectifier | 58.5816 | 41.6688 | 64.1636 |
| | potassium channel 10 | | | |
| Kcnj2 | inward rectifier potassium | 3.32953 | 2.1617 | 3.20513 |
| | channel 2 | | | |
| Kif9 | kinesin-like protein KIF9 isoform | 1.59288 | 3.60591 | 1.8759 |
| | 1 | | | |
| Kit | mast/stem cell growth factor | 19.5399 | 30.3057 | 18.7665 |
| | receptor precursor | | | |
| Klhdc7a | kelch domain-containing protein | 4.08318 | 6.5124 | 4.61306 |
| | 7A | | | |
| Lama5 | laminin subunit alpha-5 precursor | 0.800781 | 1.31662 | 0.886869 |
| Lamp2 | lysosome-associated membrane | 32.4913 | 56.8491 | 40.9038 |
| | glycoprotein 2 isoform 2 | | | |
| | precursor | | | |
| Lct | lactase-phlorizin hydrolase | 9.33636 | 5.73069 | 10.7663 |
| | preproprotein | | | |
| Leprel4 | synaptonemal complex protein | 9.45108 | 13.3996 | 7.90701 |
| | SC65 | | | |
| Lingo1 | leucine rich repeat and Ig domain | 89.1295 | 115.554 | 88.754 |
| | containing 1 precursor | | | |
| Llgl2 | lethal(2) giant larvae protein | 0.202787 | 0.963496 | 0.437277 |
| | homolog 2 | | | |
| Lmx1a | LIM homeobox transcription | 0.252613 | 1.06817 | 0.503385 |
| | factor 1-alpha | | | |
| Loxl1 | lysyl oxidase homolog 1 | 1.43382 | 2.72047 | 1.48647 |
| | precursor | | | |
| Loxl2 | lysyl oxidase homolog 2 | 0.27556 | 0.612723 | 0.191226 |
| | precursor | | | |
| Lrp10 | low-density lipoprotein receptor- | 14.2581 | 20.874 | 14.6733 |
| | related protein 10 precursor | | | |
| Lrp5 | low-density lipoprotein receptor- | 2.44877 | 3.69413 | 2.43168 |
| | related protein 5 precursor | | | |
| Ltc4s | leukotriene C4 synthase | 5.52378 | 16.1504 | 7.10216 |
| Lypd1 | ly6/PLAUR domain-containing | 49.6208 | 34.7748 | 47.9728 |
| | protein 1 precursor | | | |
| Mccc1 | methylcrotonoyl-CoA | 5.24279 | 8.91975 | 5.41031 |
| | carboxylase subunit alpha, | | | |
| | mitochondrial | | | |
| Mfsd7c | feline leukemia virus subgroup C | 0.719071 | 1.70601 | 0.649977 |
| | receptor-related protein 2 | | | |
| Mmp15 | matrix metallopeptidase 15 | 6.00865 | 9.13217 | 6.69899 |
| | precursor | | | |
| Mpp7 | MAGUK p55 subfamily member | 0.769994 | 2.31846 | 1.16242 |
| | 7 isoform 2 | | | |
| Myoc | myocilin precursor | 12.4547 | 7.92294 | 11.9051 |
| Myof | myoferlin | 0.592499 | 1.93846 | 0.862876 |
| Ndst4 | bifunctional heparan sulfate N- | 5.91472 | 3.53341 | 5.12937 |
| | deacetylase/N-sulfotransferase 4 | | | |
| Nek11 | serine/threonine-protein kinase | 0.411956 | 1.29101 | 0.580058 |
| | Nek11 | | | |
| Nid2 | nidogen-2 precursor | 0.600483 | 2.68029 | 1.14147 |
| Nos1 | nitric oxide synthase, brain | 9.09696 | 12.1959 | 8.7895 |
| Npas4 | neuronal PAS domain-containing | 4.00469 | 1.85785 | 3.8842 |
| | protein 4 | | | |
| Npr1 | atrial natriuretic peptide receptor | 1.39849 | 2.76668 | 1.7036 |
| | 1 precursor | | | |
| Npr3 | atrial natriuretic peptide receptor | 4.43706 | 7.71012 | 4.77945 |
| | 3 isoform a precursor | | | |
| Nqo1 | NAD(P)H dehydrogenase | 3.82878 | 6.31871 | 3.79412 |
| | [quinone] 1 | | | |
| Nt5dc1 | 5′-nucleotidase domain- | 0.941295 | 2.18533 | 1.20464 |
| | containing protein 1 | | | |
| Ntn4 | netrin 4 precursor | 2.25261 | 4.49753 | 2.7778 |
| Oca2 | P protein | 0.198144 | 1.52442 | 0.466956 |
| Odz4 | teneurin-4 | 7.93472 | 11.6456 | 7.19365 |
| Ooep | oocyte-expressed protein | 0.0563111 | 1.15859 | 0.0720188 |
| | homolog | | | |
| Pbxip1 | pre-B-cell leukemia transcription | 15.8609 | 21.5464 | 16.2733 |
| | factor-interacting protein 1 | | | |
| Pcp4l1 | Purkinje cell protein 4-like | 41.1701 | 70.5608 | 50.8954 |
| | protein 1 | | | |
| Pgcp | carboxypeptidase Q precursor | 6.29978 | 14.9086 | 8.02009 |
| Phactr2 | phosphatase and actin regulator 2 | 5.17232 | 9.12477 | 6.29506 |
| Pla2g4e | cytosolic phospholipase A2 | 2.12889 | 0.884548 | 1.84977 |
| | epsilon | | | |
| Plek2 | pleckstrin-2 | 0.203647 | 1.46827 | 0.430062 |
| Plekha2 | pleckstrin homology domain- | 10.3381 | 7.74972 | 12.177 |
| | containing family A member 2 | | | |
| Pltp | phospholipid transfer protein | 32.0432 | 56.2175 | 36.9312 |
| | precursor | | | |
| Plxnb2 | plexin-B2 precursor | 8.99607 | 15.0115 | 10.316 |
| Polr1a | DNA-directed RNA polymerase I | 4.88718 | 7.619 | 5.4566 |
| | subunit RPA1 | | | |
| Pon1 | serum paraoxonase/arylesterase 1 | 0 | 0.874886 | 0.119983 |
| | precursor | | | |
| Ppfibp2 | protein tyrosine phosphatase, | 1.94608 | 4.25408 | 2.52966 |
| | receptor-type, F interacting | | | |
| | protein, binding protein 2 | | | |
| Ppp1r1a | protein phosphatase 1 regulatory | 70.8683 | 47.2999 | 67.7123 |
| | subunit 1A | | | |
| Ppp1r1b | protein phosphatase 1 regulatory | 32.6242 | 61.8645 | 37.5707 |
| | subunit 1B | | | |
| Prelp | prolargin precursor | 7.6691 | 15.2924 | 9.53012 |
| Prox1 | prospero homeobox protein 1 | 15.7074 | 10.5301 | 14.3616 |
| Prps2 | ribose-phosphate | 9.91539 | 17.8129 | 12.0363 |
| | pyrophosphokinase 2 | | | |
| Ptpn14 | tyrosine-protein phosphatase non- | 1.65351 | 2.28015 | 1.31521 |
| | receptor type 14 | | | |
| Rab11fip1 | rab11 family-interacting protein 1 | 0.418415 | 1.42688 | 0.698949 |
| | isoform 2 | | | |
| Rab20 | ras-related protein Rab-20 | 0.673078 | 4.76363 | 1.27281 |
| Rai14 | ankycorbin | 1.00592 | 1.85296 | 1.05342 |
| Rbp3 | retinol-binding protein 3 | 0.279339 | 0.0795398 | 0.52036 |
| | precursor | | | |
| Rd3 | protein RD3 isoform 2 | 0.292228 | 1.51799 | 0.665572 |
| Ripk4 | receptor-interacting | 0.200004 | 0.688755 | 0.15646 |
| | serine/threonine-protein kinase 4 | | | |
| Robo3 | roundabout homolog 3 | 2.91933 | 1.69267 | 2.95522 |
| Rrh | visual pigment-like receptor | 0.0537773 | 0.750069 | 0.0523507 |
| | peropsin | | | |
| Rsph4a | radial spoke head protein 4 | 2.15178 | 4.68256 | 2.9407 |
| | homolog A | | | |
| Scg5 | neuroendocrine protein 7B2 | 209.728 | 129.591 | 219.826 |
| | precursor | | | |
| Scn4b | sodium channel subunit beta-4 | 7.1377 | 5.17018 | 8.64274 |
| | precursor | | | |
| Scube1 | signal peptide, CUB and EGF- | 4.37878 | 6.16819 | 4.58617 |
| | like domain-containing protein 1 | | | |
| | precursor | | | |
| Scube3 | signal peptide, CUB and EGF- | 0.428595 | 1.94397 | 0.628057 |
| | like domain-containing protein 3 | | | |
| | precursor | | | |
| Sdk1 | protein sidekick-1 | 0.680194 | 1.1381 | 0.682146 |
| Serinc2 | serine incorporator 2 precursor | 2.92189 | 4.9039 | 2.1423 |
| Serpinb1b | leukocyte elastase inhibitor B | 0.425173 | 2.42895 | 0.916896 |
| Sfrp1 | secreted frizzled-related protein 1 | 1.50564 | 8.21435 | 2.22656 |
| | precursor | | | |
| Sfrp5 | secreted frizzled-related protein 5 | 0.124305 | 4.32991 | 0.538409 |
| | precursor | | | |
| Sh3d19 | SH3 domain-containing protein | 3.29706 | 7.26515 | 4.4249 |
| | 19 | | | |
| Slc12a2 | solute carrier family 12 member 2 | 16.1979 | 29.0989 | 20.5749 |
| Slc12a4 | solute carrier family 12 member 4 | 5.46407 | 8.69934 | 5.89774 |
| | isoform 1 | | | |
| Slc12a7 | solute carrier family 12 member 7 | 0.837265 | 1.88202 | 1.124 |
| Slc16a12 | monocarboxylate transporter 12 | 0.751866 | 2.99108 | 1.22643 |
| Slc16a2 | monocarboxylate transporter 8 | 13.9329 | 27.5623 | 14.2113 |
| Slc16a4 | monocarboxylate transporter 5 | 1.40996 | 4.19366 | 2.15677 |
| Slc16a9 | monocarboxylate transporter 9 | 0.936307 | 3.01809 | 1.38469 |
| Slc22a6 | solute carrier family 22 member 6 | 0.781701 | 0.307055 | 0.787313 |
| Slc23a2 | solute carrier family 23 member 2 | 25.4985 | 35.3626 | 27.4105 |
| Slc25a39 | solute carrier family 25 member | 30.328 | 41.6143 | 30.7081 |
| | 39 | | | |
| Slc28a3 | solute carrier family 28 member 3 | 0.0253638 | 0.532495 | 0.118665 |
| Slc29a4 | equilibrative nucleoside | 10.2431 | 23.2085 | 11.9409 |
| | transporter 4 | | | |
| Slc37a2 | sugar phosphate exchanger 2 | 0.460742 | 1.93774 | 0.838691 |
| Slc39a4 | zinc transporter ZIP4 precursor | 0.776647 | 2.7583 | 1.07306 |
| Slc4a10 | sodium-driven chloride | 43.9167 | 63.0669 | 46.0086 |
| | bicarbonate exchanger | | | |
| Slc5a3 | solute carrier family 5 (inositol | 4.43941 | 7.60522 | 5.28894 |
| | transporters), member 3 | | | |
| Slc7a3 | cationic amino acid transporter 3 | 1.24657 | 2.85656 | 1.49931 |
| Slco1c1 | solute carrier organic anion | 17.1329 | 32.9264 | 20.1851 |
| | transporter family member 1C1 | | | |
| Smpdl3a | acid sphingomyelinase-like | 19.0378 | 27.9583 | 19.7502 |
| | phosphodiesterase 3a precursor | | | |
| Sntb1 | beta-1-syntrophin | 1.37431 | 2.97935 | 1.26937 |
| Sod3 | extracellular superoxide | 4.35475 | 9.76844 | 6.09994 |
| | dismutase [Cu—Zn] precursor | | | |
| Spag16 | sperm-associated antigen 16 | 0.279303 | 1.28905 | 0.460786 |
| | protein | | | |
| Spint2 | serine protease inhibitor, Kunitz | 10.8856 | 44.8051 | 15.9511 |
| | type 2 isoform a precursor | | | |
| Sptlc3 | serine palmitoyltransferase 3 | 0.13799 | 0.774689 | 0.23455 |
| Ssfa2 | sperm-specific antigen 2 homolog | 9.69564 | 12.9974 | 10.0587 |
| St6galnac2 | alpha-N-acetylgalactosaminide | 1.13226 | 4.6868 | 2.04296 |
| | alpha-2,6-sialyltransferase 2 | | | |
| Stk39 | STE20/SPS1-related proline- | 23.4886 | 42.4337 | 28.2125 |
| | alanine-rich protein kinase | | | |
| Stra6 | stimulated by retinoic acid gene 6 | 3.68191 | 7.80541 | 4.81665 |
| | protein | | | |
| Tbc1d1 | TBC1 domain family member 1 | 4.82631 | 2.96943 | 4.99315 |
| Tbc1d2 | TBC1 domain family member 2A | 0.601313 | 2.11639 | 0.827272 |
| Tbc1d9 | TBC1 domain family member 9 | 18.7417 | 33.3274 | 19.741 |
| Tbcel | tubulin-specific chaperone | 16.9354 | 21.9454 | 17.0147 |
| | cofactor E-like protein | | | |
| Tcn2 | transcobalamin-2 precursor | 6.71687 | 17.0888 | 9.13912 |
| Tead1 | transcriptional enhancer factor | 3.68467 | 5.57789 | 4.07515 |
| | TEF-1 isoform 2 | | | |
| Tgfb2 | transforming growth factor beta-2 | 16.9026 | 28.1203 | 21.5647 |
| | precursor | | | |
| Tgfbi | transforming growth factor-beta- | 1.02606 | 2.58149 | 1.20228 |
| | induced protein ig-h3 precursor | | | |
| Tgfbr3 | transforming growth factor beta | 2.97682 | 5.77849 | 3.87974 |
| | receptor type 3 precursor | | | |
| Timp2 | metalloproteinase inhibitor 2 | 81.4295 | 120.556 | 84.9023 |
| | precursor | | | |
| Tinagl1 | tubulointerstitial nephritis | 2.45869 | 5.00213 | 2.32016 |
| | antigen-like precursor | | | |
| Tjp3 | tight junction protein ZO-3 | 0.625603 | 1.99657 | 0.951327 |
| Tlr2 | toll-like receptor 2 precursor | 0.621807 | 1.96697 | 1.03535 |
| Tmed3 | transmembrane emp24 domain- | 17.2348 | 26.1727 | 18.8052 |
| | containing protein 3 precursor | | | |
| Tmem108 | transmembrane protein 108 | 8.20673 | 11.1922 | 8.4143 |
| | precursor | | | |
| Tmem27 | collectrin precursor | 0.037105 | 1.14839 | 0.365653 |
| Tmem98 | transmembrane protein 98 | 8.92329 | 18.9354 | 10.1715 |
| Tns1 | tensin 1 | 3.9512 | 5.81114 | 3.86792 |
| Tspan33 | tetraspanin-33 | 14.3433 | 23.7053 | 15.0442 |
| Ttc21a | tetratricopeptide repeat protein | 0.690266 | 1.89625 | 1.10696 |
| | 21A | | | |
| Tuft1 | tuftelin | 0.895052 | 2.15741 | 1.14097 |
| Vamp8 | vesicle-associated membrane | 10.4466 | 24.9316 | 13.404 |
| | protein 8 | | | |
| Vcam1 | vascular cell adhesion protein 1 | 10.1223 | 13.7747 | 10.2949 |
| | precursor | | | |
| Vcp | transitional endoplasmic | 0.97429 | 2.928 | 1.54483 |
| | reticulum ATPase | | | |
| Wdfy1 | WD repeat and FYVE domain | 7.51421 | 10.171 | 7.05013 |
| | containing 1 | | | |
| Wdr16 | WD repeat-containing protein 16 | 0.854432 | 3.4711 | 1.6294 |
| Wdr72 | WD repeat domain 72 | 0.00638566 | 0.817544 | 0.18906 |
| Wfs1 | wolframin | 40.0759 | 24.9153 | 43.1125 |
| Zfp185 | zinc finger protein 185 isoform a | 0.745355 | 2.85428 | 1.3415 |
| Zfp605 | zinc finger protein 605 | 4.77237 | 8.21454 | 4.39751 |

### Example 3

Three month old, male, SXFAD mice were treated for 1 month (every other day), via intraperitoneal injections with the histone deacetylase inhibitor; CI-994 (1 mg/kg), which has been shown to reduce AD-related cognitive impairments. After completion of treatment, blood was drawn and peripheral blood mononuclear cells were rapidly isolated. The cells were washed with PBS and total RNA was extracted using the RNeasy kit (Qiagen). RNA integrity was analyzed using the Bioanalyzer 2100 (Agilent) and the libraries were prepared using the Ovation Ultralow Library System kit (NuGen). Libraries were then pooled in equal amounts and high-throughput sequencing was performed on an Illumina HiSeq 2000 platform. Two individual biological replicates per condition were sequenced.

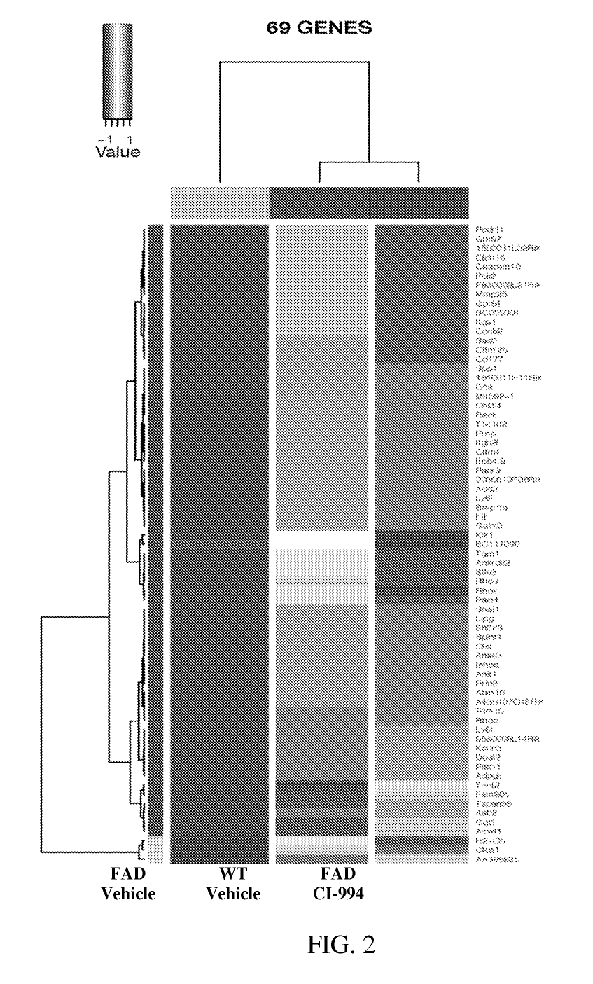

69 genes were found to be differentially expressed between wild type and SXFAD mice, which could be rescued to control levels with CI-994 treatment (FIG. 2). This result suggests pathological changes in the brain are reflected in the blood (via PBMCs) and HDAC inhibitors can not only reverse these changes but this rescue can be detected in circulating blood cells.

Moreover, 18 genes (Table 2) that are upregulated in the SXFAD blood samples, were also upregulated in the SXFAD brain samples. Of the 18 genes, three genes; Tbc1d2, Tspan33, and Kit, are rescued with CI-994 treatment in both the brain and blood samples.

TABLE 2
A list of 18 genes that are differentially expressed between 5XFAD
mice and littermate controls. Shown below are a list of 18 differentially
expressed genes identified by RNA-sequencing of PBMCs and brain
lysates. Gene differential analysis was performed by using Cuffdiff
(Trapnell et al., 2013) with Refseq gene database provided by Illumina.
A gene was considered differentially expressed with a fold change
of ≧1.4 and a significance of p ≦ 0.05.

| Gene Name | Chromosome Locus | p-value |
|---|---|---|
| Cdr2 | chr7: 128100549-128125826 | 1.88E−08 |
| Stk39 | chr2: 68048503-68310038 | 8.72E−07 |
| Tbc1d2 | chr4: 46617261-46663071 | 2.19E−06 |
| Bmp7 | chr2: 172695188-172765794 | 4.33E−05 |
| Nsdhl | chrX: 70163859-70203867 | 5.93E−05 |
| Lbp | chr2: 158132228-158158588 | 0 |
| Tspan33 | chr6: 29644255-29668558 | 0.000241 |
| Cish | chr9: 107199019-107204292 | 0.00077 |
| Fam46c | chr3: 100275458-100293115 | 0.000772 |
| Ctsl | chr13: 64464521-64471614 | 0.002141 |
| Kit | chr5: 75971011-76052746 | 0.002661 |
| Crtac1 | chr19: 42357526-42506273 | 0.012079 |
| Emilin1 | chr5: 31216158-31223646 | 0.021279 |
| Pafah2 | chr4: 133952274-133983327 | 0.027017 |
| Nqo1 | chr8: 109912124-109927105 | 0.029268 |
| Ptprf | chr4: 117880817-117964002 | 0.031627 |
| Ttc12 | chr9: 49245065-49294330 | 0.047612 |

TABLE 3
List of 69 differentially expressed genes

| Gene | Full name |
|---|---|
| Podnl1 | Podocan-Like 1 |
| Gpr97 | G Protein-Coupled Receptor 97 |
| 1500031L02Rik | Cep19 centrosomal protein 19 |
| Cldn15 | Claudin 15 |
| Ceacam10 | Carcinoembryonic antigen-related cell |
| | adhesion molecule 10 |
| Peli2 | Pellino E3 Ubiquitin Protein Ligase Family |
| | Member 2 |
| F830002L21Rik | RIKEN cDNA F830002L21 gene |
| Mmp25 | matrix metallopeptidase 25 |
| Gpr84 | G protein-coupled receptor 84provided |
| BC055004 | Nxpe5 neurexophilin and PC-esterase |
| | domain family, member 5 |
| Itga1 | integrin, alpha 1 |
| Ccnb2 | cyclin B2 |
| Saa3 | serum amyloid A 3 |
| Olfml2b | olfactomedin-like 2B |
| Cd177 | CD177 molecule |
| Spp1 | secreted phosphoprotein 1 |
| 1810011H11Rik | RIKEN cDNA 1810011H11 gene |
| | (1810011H11Rik), mRNA |
| Gca | grancalcin, EF-hand calcium binding |
| | protein |
| Mir692-l | microRNA 692-1 |
| CM3l4 | chitinase 3-like 4 |
| Reck | reversion-inducing-cysteine-rich protein |
| | with kazal motifs |
| Tbc1d2 | TBC1 domain family, member 2 |
| Prnp | prion protein |
| Itgb2l | integrin beta 2-like |
| Olfm4 | olfactomedin 4 |
| Epb4.9 | erythrocyte protein band 4.9 |
| Paqr9 | progestin and adipoQ receptor family |
| | member IX |
| 9030619P08Rik | RIKEN cDNA 9030619P08 gene |
| Add2 | adducin 2 (beta) |
| Ly6i | lymphocyte antigen 6 complex, locus I |
| Bmpr1A | bone morphogenetic protein receptor, type |
| | 1A |
| Kit | kit oncogene |
| Galnt3 | UDP-N-acetyl-alpha-D- |
| | galactosamine: polypeptide N- |
| | acetylgalactosaminyltransferase 3 |
| | (GalNAc-T3) |
| Klk1 | kallikrein 1 |
| BC117090 | cDNA sequence BC1179090 |
| Tgm1 | transglutaminase 1 |
| Ankrd22 | ankyrin repeat domain 22 |
| Stfa3 | stefin A3 |
| Rhou | ras homolog family member U |
| Rhov | ras homolog family member V |
| Padi4 | peptidyl arginine deiminase, type IV |
| Snai1 | snail family zinc finger 1 |
| Lipg | lipase, endothelial |
| Sh3rf3 | SH3 domain containing ring finger 3 |
| Spint1 | serine peptidase inhibitor, Kunitz type 1 |
| Ctsl | cathepsin |
| Anxa3 | annexin A3 |
| Inhba | inhibin, beta A |
| Ank1 | ankyrin 1, erythrocytic |
| Prtn3 | proteinase 3 |
| Atxn10 | ataxin 10 |
| A430107O13Rik (Cped1) | Cped1 cadherin-like and PC-esterase |
| | domain containing 1 |
| Trim10 | tripartite motif containing 10 |
| Rhoc | ras homolog family member C |
| Ly6f | Ly6f lymphocyte antigen 6 complex, locus |
| | F |
| 9530008L14Rik | RIKEN cDNA 9530008L14 gene |
| Kcnn3 | potassium intermediate/small conductance |
| | calcium-activated channel, subfamily N, |
| | member 3 |
| Dgat2 | diacylglycerol O-acyltransferase 2 |
| Plscr1 | phospholipid scramblase 1 |
| Adpgk | ADP-dependent glucokinase |
| Tnnt2 | troponin T type 2 (cardiac) |
| Fam20c | family with sequence similarity 20, |
| | member C |
| Tspan33 | tetraspanin 33 |
| Asb2 | ankyrin repeat and SOCS box containing 2 |
| Ggt1 | gamma-glutamyltransferase 1 |
| Acvrl1 | activin A receptor type II-like 1 |
| H20Ob | histocompatibility 2, O region beta locus |
| Clca1 | chloride channel accessory 1 |
| AA388235 | expressed sequence AA388235 |

We claim:

1. A method of assessing the efficacy of a putative therapy for Alzheimer's disease in a subject in need thereof comprising:
(a) administering the putative therapy to the subject to treat the Alzheimer's disease;
(b) measuring an expression pattern of at least one Alzheimer's disease-associated gene in an isolated biological sample from the subject; and
(c) comparing the expression pattern with an appropriate reference expression pattern of the at least one Alzheimer's disease-associated gene, wherein the results of the comparison are indicative of the efficacy of the putative therapy.

2. A method comprising:
performing an assay to measure an expression pattern of at least one Alzheimer's disease-associated gene in an isolated biological sample from a subject; and
comparing the expression pattern with an appropriate reference expression pattern of the at least one Alzheimer's disease-associated gene, wherein the results of the comparison are indicative of the presence of an Alzheimer's disease phenotype in the subject.

3. The method of claim 2, wherein the expression pattern of at least 5, at least 10, at least 25, at least 50, at least 75, at least 100, at least 125, at least 150, at least 175, at least 200, at least 225, at least 250 Alzheimer's disease-associated genes is measured, and compared to the appropriate reference expression pattern.

4. The method of claim 2, wherein the biological sample is selected from the group consisting of blood, serum, cerebrospinal fluid, urine and tissue.

5. The method of claim 2, wherein the appropriate reference expression pattern comprises:
(i) expression levels of the Alzheimer's disease-associated genes in a biological sample obtained from a subject who does not have Alzheimer's disease;
(ii) expression levels of the Alzheimer's disease-associated genes in a biological sample obtained from the subject prior to treatment; and
(iii) standard expression levels of the Alzheimer's disease-associated genes.

6-9. (canceled)

10. The method of claim 2, wherein the Alzheimer's associated genes comprise genes selected from Tables 1, 2 and/or 3

11. The method of claim 2, wherein the Alzheimer's associated genes comprise Tbc1d2, Tspan33, and/or Kit.

12. The method of claim 2, wherein the expression pattern of RNA encoded by the Alzheimer's disease associated genes is measured using a hybridization-based assay.

13. (canceled)

14. The method of claim 2, wherein the expression pattern of proteins encoded by the Alzheimer's disease associated genes is measured using an antibody-based assay.

15. (canceled)

16. The method of claim 1, wherein the putative therapy is an HDAC inhibitor.

17. The method of claim 2, wherein the method is a method of monitoring progression of Alzheimer's disease in a subject in need thereof and wherein the method further comprises:
(a) obtaining a first biological sample from the subject;
(b) measuring a first expression pattern of at least one Alzheimer's disease-associated gene in the biological sample;
(c) obtaining a second biological sample from the subject;
(d) measuring a second expression pattern of the at least one Alzheimer's disease-associated gene in the biological sample;
(e) comparing the first expression pattern with the second expression pattern, wherein the results of the comparison are indicative of the extent of progression of Alzheimer's disease in the subject.

18. The method of claim 17, wherein between obtaining the first biological sample and obtaining the second biological sample, the subject is treated with HDAC inhibitor therapy.

19. (canceled)

20. The method of claim 2, wherein the method is a method for identifying a therapy for the subject, and wherein the method involves selecting an HDAC inhibitor as a therapy for the subject if the Alzheimer's disease associated gene that is modulated is a gene from Table 2 or 3.

21. The method of claim 20, further comprising treating the subject with an HDAC inhibitor.

22. (canceled)

23-26. (canceled)

27. The method of claim 2, wherein the at least Alzheimer's associated gene is
wherein a lower level of beta-amyloid protein in the biological sample in comparison to a reference level associated with a normal subject is indicative of the presence of an Alzheimer's disease phenotype in the subject, and wherein the biological sample is a tissue other than the brain.

28. The method of claim 27, wherein the biological sample is cerebrospinal fluid, blood or plasma.

29-31. (canceled)

32. A method of treating an inflammatory disorder of the brain or CNS
administering to a subject an inflammatory disorder of the brain or CNS which is a non-cognitive neurodegenerative disorder an HDAC inhibitor in an effective amount to treat the disorder.

33. The method of claim 32, further comprising performing an assay to measure an expression pattern of at least one Alzheimer's disease-associated gene in an isolated biological sample from a subject; and comparing the expression pattern with an appropriate reference expression pattern of the at least one Alzheimer's disease-associated gene, wherein the results of the comparison are indicative of the effectiveness of treating the disorder with an HDAC inhibitor.

34. The method of claim 32, wherein the HDAC inhibitor is a specific HDAC 1, HDAC 2 and/or HDAC3 inhibitor.

35. The method of claim 32, wherein the HDAC inhibitor is CI-994.

36-37. (canceled)
